(19) United States
(12) Patent Application Publication
(10) Pub. No.: US 2025/0066700 A1
(43) Pub. Date: Feb. 27, 2025

(54) METHOD FOR PRODUCING A FERMENTED GRAPE MARC, RESULTING FERMENTED GRAPE MARC AND USE THEREOF IN DIETARY SUPPLEMENTS OR IN COSMECEUTICALS

(71) Applicants: INSTITUT NATIONAL POLYTECHNIQUE DE TOULOUSE, TOULOUSE (FR); UNIVERSITE TOULOUSE III – PAUL SABATIER, TOULOUSE (FR); CENTRE NATIONAL DE LA RECHERCHE SCIENTIFIQUE, PARIS (FR)

(72) Inventors: Patricia TAILLANDIER, TOULOUSE CEDEX 4 (FR); Sandra BEAUFORT, TOULOUSE CEDEX 4 (FR); Jalloul BOUAJILA, TOULOUSE CEDEX 4 (FR); Agathe JUPPEAU, TOULOUSE CEDEX 4 (FR); Isaura CACERES, TOULOUSE CEDEX 4 (FR)

(73) Assignees: INSTITUT NATIONAL POLYTECHNIQUE DE TOULOUSE, TOULOUSE (FR); UNIVERSITE TOULOUSE III – PAUL SABATIER, TOULOUSE (FR); CENTRE NATIONAL DE LA RECHERCHE SCIENTIFIQUE, PARIS (FR)

(21) Appl. No.: 18/720,854

(22) PCT Filed: Dec. 15, 2022
(86) PCT No.: PCT/FR2022/052362

§ 371 Date: Jun. 17, 2024

(30) Foreign Application Priority Data
Dec. 16, 2021 (FR) FR2113743

(51) Int. Cl. C12G 1/028

(52) U.S. Cl. CPC C12G 1/0213

(57) ABSTRACT

A method for preparing a fermented grape marc, which includes the following steps: preparing a grape marc infusion by placing grape marc in contact with water and optionally with a source of sugar chosen among sucrose, glucose syrup, or concentrated grape must; placing the grape marc infusion in contact with a scoby (also called “kombucha microbial consortium”) and optionally with kombucha; fermenting the grape marc infusion for a period ranging from 5 to 35 days, and preferably from 7 to 12 days. Also, fermented grape marc obtainable according to the method, as well as to the use thereof in dietary, nutraceutical, or nutricosmetic supplements, or in dermocosmetic products.

### FIELD

This invention relates to the field of health. In particular, it concerns dietary or nutraceutical supplements as well as cosmeceuticals comprising fermented grape marc. This invention also relates to a method for fermenting grape marc using a microbial consortium from kombucha (SCOBY).

### BACKGROUND

The global market is booming for dietary supplements, also known as food supplements, with growth expected to exceed 100% by 2025, in particular due to the growing awareness of the link between health and food.

According to the definition given by Directive 2002/46/EC of the European Parliament, “food supplements means foodstuffs the purpose of which is to supplement the normal diet and which are concentrated sources of nutrients or other substances with a nutritional or physiological effect, alone or in combination, marketed in dose form”.

The word “nutraceutical”, for its part, does not have an official definition but comes from the contraction of “nutrition” and “pharmaceutical”. In other words, the terms “nutraceuticals” and “dietary supplements” overlap.

Dietary supplements are also of interest to cosmetic laboratories, with the emergence of “cosmeceuticals” and more particularly of “dermocosmetics” and “nutricosmetics” and the “in & out” trend defined by the importance of nourishing the skin from the inside. The word “cosmeceutical,” which is a portmanteau word combining “cosmetic” and “pharmaceutical,” refers to a cosmetic product containing a bioactive ingredient believed to have medical benefits. However, there is no legal requirement to prove that these products live up to their claims. Nutricosmetics (a word combining cosmetics and nutraceuticals) correspond to edible products, while dermocosmetic products are applied locally to the skin, scalp, hair, etc.

Presented in dose form, food or nutraceutical supplements exist in different forms of administration and can be composed of various ingredients: vitamins and minerals, substances for nutritional or physiological purposes (amino acids, omega-3, etc.), plant extracts or metabolites, ingredients of animal origin (fish oils, propolis, etc.), ferments and yeasts (probiotics, brewer's yeast, etc.), fruits and vegetables.

Dietary supplements lie somewhere between food that nourishes and medicine that treats. Depending on their composition, their role may be nutritional or physiological. Thus, certain dietary supplements, given their high concentration of nutrients, make it possible to compensate for dietary deficiencies, and in particular to meet specific needs of certain populations (pregnant women, elderly people, etc.) or in certain situations (vitamin D deficiency in winter, vitamin B12 deficiency, etc). In other cases, dietary supplements help reduce or alleviate discomfort in healthy people (stress, joint pain, difficulty sleeping, etc.). Dietary supplements also have a preventive role, by reducing the risk factors for chronic diseases (lowering cholesterol, strengthening bone mineral density, etc.). The various active ingredients present in dietary supplements will allow physiological equilibrium and thus keep consumers in good health.

Given the growing importance of this market, there is still a need to develop new dietary supplements or cosmeceuticals. In this regard, the inventors took particular interest in grape marc.

Grape marc is present in many dietary supplements and is particularly popular in cosmetics and herbal medicine. It has a wide variety of polyphenols (flavonoids, anthocyanins, tannins, phenolic acids, stilbenes, etc.) and is mainly known for its anti-oxidant and draining properties.

It has also been described as exhibiting anti-inflammatory properties.

Grape marc is a by-product obtained during wine production.

In the manufacture of white wine, the first step is to crush or press entire grape bunches which allows obtaining grape must, a thick and sweet unfermented grape juice. The stems, skins, seeds, stems, etc. are removed from this thick juice, i.e. everything solid that remains after pressing the grapes. The liquid part thus obtained is used to make wine while the solid part (=residue from the must) constitutes the grape marc.

In the production of red wine, once the grapes are crushed, they are placed in vats for maceration. During maceration, the solid parts (skins, pulp, seeds) rise to the surface and form the “marc cap”. It is also during maceration that alcoholic fermentation takes place. To end the maceration, the juice is collected from the bottom of the tank in order to separate it from the solid parts (the marc cap).

Whether for white wine or red wine, grape marc refers to all the solid residues obtained after the grapes are pressed, whether before or after alcoholic fermentation. Its appearance is dry and crumbly and it has a color that varies between yellow and purple depending on the grape varieties used.

Kombucha is a traditional, slightly acidic drink originating from Asia, believed to have beneficial effects on health. Kombucha consists of a sweet tea that has been fermented by a mixed population of bacteria and yeast, called a “microbial consortium.” Microbial consortium refers more particularly to a mixture of microorganisms, namely bacteria and yeasts, which live in symbiosis. Microbial consortium is further defined by the acronym “SCOBY” (referred to as scoby in the following), for “symbiotic culture of bacteria and yeasts”.

To prepare kombucha, scoby is placed in contact with a tea (black tea or green tea, either will do) which has been previously sweetened. The scoby yeasts and bacteria will develop in the sweetened tea and enable alcoholic fermentation and acetic acid fermentation respectively. In short, the scoby yeasts use the sugar to produce alcohol and carbon dioxide, and the scoby bacteria use the produced alcohol to produce acetic acid. At the same time, the acetic bacteria also synthesize sugars from the cellulose which is primarily located in the surface film of the scoby. Many metabolites of interest are also produced by the scoby microorganisms, such as vitamins, gluconic acid, glucuronic acid, etc.

In its most common form, scoby has the appearance of a gelatinous film/membrane that is several centimeters thick. When scoby is placed in contact with tea, it generally floats at the tea-air interface of the container containing the tea. During fermentation, scoby adapts to the size of the container so as to cover the entire surface of the tea. The length of fermentation is approximately 10 to 15 days at room temperature. At the end of fermentation, a new scoby has formed across the entire tea-air interface. The scoby used at the start of fermentation is called the “kombucha mother” while the scoby formed at the end of fermentation is called the “kombucha baby”. The kombucha baby forms above the kombucha mother (in fact the new film/membrane grows at the interface with the air). It is easy to separate the kombucha baby from the kombucha mother (e.g. manually). The kombucha baby can in turn be used, alone or in association with the kombucha mother, to initiate a new fermentation of a sweetened tea and will thus become a kombucha mother.

Like all living organisms, SCOBYs resemble one another but are not identical. Inhabited by millions of micro-organisms, their composition of bacteria and yeasts can vary (in particular depending on the regions, the temperature, the tea used, the size of the reactor, etc.) and give a different result for the kombucha obtained.

### SUMMARY

One of the aims of the present invention is to develop new dietary supplements or cosmeceuticals based on grape marc.

In this regard, the Inventors had two original ideas, namely:
preparing fermented grape marc,
using a kombucha microbial consortium to ferment the grape marc.

Indeed, the Inventors hypothesized that a kombucha microbial consortium could be capable of transforming the compounds of interest that are present in grape marc, in order to potentiate their biological properties.

Another aim of the invention is therefore to develop new dietary supplements or cosmeceuticals based on fermented grape marc.

Yet another aim of the invention is to develop a method for fermenting grape marc using a kombucha microbial consortium.

As indicated above, the inventors conceived of using a kombucha microbial consortium to ferment grape marc.

However, nothing led to assume that grape marc was a fermentable substrate. Indeed, as indicated above, in the case of manufacturing red wine, the grape marc is obtained after alcoholic fermentation, which suggests that it has already been exhausted of nutrients for microorganisms (vitamins, nitrogen compounds, mineral salts, etc.) and that it may also contain fermentation inhibitors.

It is therefore to the Inventors' credit to have had the idea of using grape marc to undergo fermentation by a kombucha microbial consortium.

The present invention relates to a method for preparing fermented grape marc, characterized in that it comprises the following steps:
preparing a grape marc infusion by placing grape marc in contact with water and possibly with a source of sugar chosen among sucrose, glucose syrup, or concentrated grape must;
placing the grape marc infusion in contact with a scoby (also called “kombucha microbial consortium”) and possibly with kombucha;
fermenting the grape marc infusion for a period ranging from 5 to 35 days, and preferably from 7 to 12 days.

The grape marc used in the method of the invention may come from any type of grape variety (varieties of wine grapes or table grapes). “Grape variety” is a variety of grape vine that produces a specific grape.

Grape marc is a particularly interesting product because it is rich in polyphenols and also contains sugar, vitamin C, as well as certain B vitamins. Depending on the method used, the sugar content can be quite high, which is why it will not necessarily be required to add sugar during the step of preparing the grape marc infusion.

According to one embodiment of the invention, the grape marc comes from a black grape variety chosen from the group comprising Cabernet Franc, Cabernet Sauvignon, Malbec, Syrah, Négrette, Merlot, and Grenache Noir.

According to another embodiment of the invention, the grape marc comes from a white grape variety chosen from the group comprising Chardonnay, Sauvignon Blanc, Grenache Blanc, Chenin, Gewurztraminer, and Riesling.

According to a preferred embodiment of the invention, the grape marc comes from producers in the region historically known as Occitania, such as the varieties Cabernet franc, Cabernet Sauvignon, Malbec, and Négrette.

As indicated above, grape marc is in the form of a solid residue. When grape marc is separated from the liquid, it contains approximately 50 to 65% moisture. Such marc residue, comprising a water content of approximately 50-65% moisture, is called “fresh residue”.

Fresh grape marc residue may also be frozen, at a temperature of around −20° C. This residue is then called “frozen residue”.

It is also possible to dry fresh grape marc residue in the sun or by heat until the water content of the solid residue is approximately 10%. A marc residue with a water content of approximately 10% is called “dry residue”.

According to one embodiment of the invention, the grape marc used at the start of the method may be in the form of fresh, frozen, or dry solid residue. Placing said grape marc (in the form of fresh, frozen, or dry solid residue) in contact with water allows obtaining a grape marc infusion.

According to another advantageous embodiment of the method of the invention, the grape marc infusion is prepared according to the method comprising the following steps:
optionally, in a preliminary step, adding a source of sugar chosen among sucrose, glucose syrup, or concentrated grape must, to water previously heated to a temperature ranging from 30° C. to 40° C., in order to achieve a concentration ranging from 30 to 50 g/L of sugar per liter of water,
adding grape marc to water previously heated to a temperature ranging from 70° C. to 90° C., said water possibly being water prepared in the previous step (namely water comprising a source of sugar), so as to obtain an infusion having a concentration ranging from 20 to 150 g/L, preferably from 50 to 80 g/L, of “grape marc” dry matter per liter of water, said water optionally comprising a source of sugar,
maintaining the temperature of the infusion at a temperature ranging from 70° C. to 90° C., for a period ranging from 10 to 20 minutes,
filtering the grape marc infusion in order to separate it from the marc remaining in solid form,
cooling the filtered grape marc infusion to a temperature ranging from 20 to 35° C.,
optionally, adding to the grape marc infusion a nitrogen source chosen among an organic nitrogen salt or ammonium salt, at a quantity ranging from 0.2 to 0.8 g/L of nitrogen source per liter of grape marc infusion.

The addition of organic nitrogen could, for example, be achieved by adding yeast derivatives. The step of adding grape marc to previously heated water is preferably carried out under mechanical stirring.

According to yet another embodiment of the method of the invention, the grape marc used at the start of the method may be a grape marc extract.

The scoby is as defined above and refers to a kombucha microbial consortium. The expressions “microbial consortium from kombucha”, “kombucha microbial consortium”, “kombucha scoby”, and “scoby” are equivalent and designate a “kombucha mother” scoby and/or a “kombucha baby” scoby.

According to one embodiment of the invention, the scoby used in the method of the invention may be purchased commercially or may be as described in the Villarreal-Soto et al. document¹. The predominant bacteria present in the scoby of Villarreal-Soto et al. are Komagataeibacter species rhaeticus, Gluconacetobacter, and Gluconobacter, while the predominant yeasts are Brettanomyces and Schizosaccharomyces.

According to yet another advantageous embodiment, the scoby used in the method for preparing fermented grape marc of the invention is prepared by the Inventors. This scoby is more particularly the one obtained at the end of the method for preparing kombucha developed by the Inventors, for which the details are given in the paragraph below.

According to one advantageous embodiment of the method of the invention, in addition to the scoby which is placed in contact with the grape marc infusion, it may be advantageous to also place the grape marc infusion in contact with a kombucha. Indeed, kombucha contains yeasts and live bacteria in suspension and acidifies the medium.

“Kombucha” refers to the drink defined above, namely tea fermented with scoby. For the purposes of this application, “kombucha” without further clarification therefore always designates “tea kombucha”.

The kombucha used in the method of the invention may be purchased commercially or may also be prepared by the Inventors. The method for preparing kombucha comprises the following three steps:
preparing an infusion of sweetened tea, preferably a black tea, and even more preferably a Ceylon black tea,
bringing the sweetened tea infusion into contact with a scoby, cider vinegar, and optionally kombucha,
fermenting the sweetened tea infusion for a period of 14 to 20 days in order to obtain kombucha.

These steps were optimized by the Inventors in the manner described below.

The first step of the method, the step of preparing the sweetened tea infusion comprises the steps consisting of:
heating the required volume of water to a temperature ranging from 30 to 40° C. then adding the required amount of sucrose in order to obtain a concentration of sugar water ranging from 60 to 80 g/L, and preferably 70 g/L;
increasing the temperature of the sugar water to 70 to 90° C., then adding the required amount of tea (in solid form) in order to obtain a tea at a concentration ranging from 10 to 30 g/L;
leaving the tea to steep for 10 to 20 minutes, stirring regularly;
leaving the tea infusion to cool down to a temperature of less than or equal to 35° C.;
filtering the tea infusion to separate out the tea remaining in solid form.

In a second step of the method for preparing kombucha, the sweetened tea infusion obtained in the previous step, cider vinegar, a scoby, and optionally kombucha are then mixed in a container intended for fermentation.

The proportions of these different ingredients, in percentage by weight, are respectively as follows: tea infusion: 95%; cider vinegar: 1%; scoby: 2%; kombucha: 2%.

The addition of kombucha is optional and it was obtained commercially the first time the Inventors prepared the kombucha. However, it could also be kombucha prepared by the Inventors when the method for preparing kombucha is repeated in a second step.

The addition of cider vinegar helps lower the pH of the mixture in order to prevent microbial contamination at the start of fermentation. Subsequently, during the fermentation step the pH naturally drops, thus preventing any microorganisms external to the scoby from developing. The scoby was obtained commercially the first time the Inventors prepared the kombucha. However, it could also be scoby obtained at the end of the method for preparing kombucha when the method for preparing kombucha is subsequently repeated by the Inventors.

In the third step of the method for preparing kombucha, fermentation is carried out at a temperature ranging from 20 to 30° C., for a period of 14 to 20 days. Preferably, the fermentation period is 15 to 17 days. Preferably, fermentation is carried out protected from light.

At the end of fermentation, the kombucha is separated from the scoby. This scoby also includes the scoby formed during the fermentation stage (the kombucha baby) and the scoby which was used to initiate fermentation (the kombucha mother). As already stated, it is easy to separate the kombucha baby from the kombucha mother. The scoby used to initiate new fermentation could be the kombucha baby alone or the kombucha baby with the kombucha mother.

As indicated in the preceding paragraph, the scoby obtained at the end of the method for preparing kombucha may be used in the method for preparing fermented grape marc. Thus, according to an advantageous embodiment of the method of the invention, the scoby placed in contact with the grape marc infusion is the scoby (kombucha baby) recovered at the end of 14 to 20 days of fermentation, and preferably 15 to 17 days of fermentation.

This scoby is close to the one identified in the previously mentioned publication by Villareal-Soto et al.¹

According to another advantageous embodiment of the method for preparing fermented grape marc of the invention, the kombucha from 14 to 20 days of fermentation, and preferably from 15 to 17 days of fermentation, is also placed in contact with the grape marc infusion.

According to an advantageous embodiment of the method of the invention:
the scoby comes from a kombucha having 14 to 20 days of fermentation, preferably 15 to 17 days of fermentation,
the kombucha is a kombucha having 14 to 20 days of fermentation, preferably 15 to 17 days of fermentation,
the grape marc infusion has a concentration of “grape marc” dry matter per liter of water ranging from 20 to 150 g/L, preferably from 50 to 80 g/L, said water optionally comprising a source of sugar chosen among sucrose and/or concentrated grape must.

As for the duration of the fermentation stage of the method for preparing fermented grape marc according to the invention, it is variable because it depends both on the products used (grape marc, scoby, and optionally sugar and/or kombucha) but also on the temperature conditions, humidity conditions, etc.

According to yet another preferred embodiment, the step of fermenting the grape marc is carried out protected from light.

According to yet another preferred embodiment, the step of fermenting is carried out under static conditions, with occasional stirring.

According to another embodiment of the method of the invention, the fermentation is carried out at a temperature which varies from 20° C. to 30° C.

According to yet another embodiment of the invention, the method for preparing fermented grape marc is characterized in that, at the end of fermentation, the scoby is removed from the fermented grape marc infusion in order to recover the fermented grape marc infusion without the scoby. The fermented grape marc infusion from which the scoby has been removed is called “grape marc kombucha”.

In the present application, “Grape marc kombucha” therefore designates a grape marc infusion fermented with a scoby and from which the scoby has been removed. Grape marc kombucha will never simply be called “kombucha”, so as not to confuse it with conventional kombucha, namely tea kombucha.

As indicated above regarding the preparation of kombucha, the scoby which is removed at the end of fermentation from the fermented grape marc infusion includes the scoby formed during the fermentation stage (the kombucha baby) as well as the scoby which was used to initiate fermentation (the kombucha mother).

As an indication, the increase in mass of the scoby at the end of fermentation (compared to the mass of the scoby before the start of fermentation) is 50% to 90%.

According to another embodiment of the invention, the method is characterized in that the fermented grape marc infusion from which the scoby has been removed is centrifuged or filtered.

According to yet another embodiment of the invention, the method is characterized in that the fermented grape marc infusion from which the scoby has been removed, optionally centrifuged or filtered, is evaporated or freeze-dried in order to obtain a dry crude extract of fermented grape marc, also called “dry crude extract of grape marc kombucha”.

According to a preferred embodiment of the invention, the fermented grape marc infusion is centrifuged or filtered before being evaporated or freeze-dried.

The dry crude extracts thus obtained may in particular be preserved by freezing.

Advantageously, according to another embodiment of the invention, it is possible to perform an additional extraction of the dry crude extract obtained, whether frozen or not, in order to eliminate the remaining microorganisms originating from the scoby while preserving the biological activities of the dry crude extracts of grape marc kombucha and while increasing their effectiveness due to the concentration of active molecules.

The additional extraction of the dry crude extract obtained could for example be carried out with ethanol or with a solvent of similar polarity.

In this regard, the method of the invention is further characterized in that it may further comprise the following steps:
solubilizing the dry crude extract of grape marc kombucha in ethanol,
stirring the ethanol solution,
filtering the ethanol solution,
evaporating the filtered ethanol solution until a dry ethanolic extract is obtained, also called “dry ethanolic extract of grape marc kombucha”.

According to one embodiment of the method of the invention, the stirring is carried out for a period ranging from 40 to 80 minutes. Similarly, it may be advantageous to carry out two successive filtrations of the previously stirred ethanol solution.

The expressions “dry extracts of grape marc kombucha”, “extracts of grape marc kombucha”, “dry extracts of fermented grape marc”, or “fermented grape marc extracts”, without further clarification, refer herein to dry crude extracts or dry ethanolic extracts or both. In the present application, a dry extract therefore designates a dry crude extract and/or a dry ethanolic extract. The extracts of grape marc kombucha obtained according to the method of the invention have extremely interesting and improved biological properties compared to the biological properties of unfermented grape marc.

The fermented grape marc extracts obtained according to the method of the invention have, in particular, promising anti-oxidant, anti-inflammatory, and anti-diabetic properties.

The present invention also relates to a fermented grape marc capable of being obtained according to the preparation method of the invention.

In this application, “fermented grape marc” designates:
a fermented grape marc infusion from which the scoby has not been removed,
grape marc kombucha (i.e. the fermented grape marc infusion from which the scoby has been removed), and/or,
dry extracts (crude or ethanolic) of fermented grape marc.

The properties highlighted by the Inventors for fermented grape marc (namely anti-oxidant, anti-inflammatory, and anti-diabetic properties) make them of particular interest for their use in dietary supplements or nutraceuticals but also in the field of cosmetics, and more particularly in nutricosmetics or in dermocosmetic products.

Thus, another object of the invention lies in the use of a grape marc obtainable or obtained according to the preparation method of the invention, in dietary supplements, said dietary supplements able to be in the form of a solid or a liquid.

As an example of a dietary supplement in solid form, one can list tablets, capsules, gelcaps, sachets, powders, etc.

As an example of a dietary supplement in liquid form, one can list drinkable solutions, syrups, ampoules, drinkable single doses, powders for dilution, but also grape marc kombucha drinks.

Yet another object of the invention lies in the use of a grape marc obtainable or obtained according to the preparation method of the invention, in nutricosmetics or in dermocosmetic products.

Nutricosmetics are more specifically defined as dietary supplements absorbed orally in order to improve the appearance and health of skin, nails, and hair. They may be in the same forms as a dietary supplement (solid or liquid form).

Dermocosmetic products, as their name suggests, are found in a form suitable for cutaneous application (skin, scalp, hair, etc.). They may be in the form of a cream, a gel, a lotion, a shampoo, etc.

When fermented grape marc is used in dietary, nutraceutical, or nutricosmetic supplements or in dermocosmetic products, it may be further combined with other ingredients such as vitamins, minerals, fatty acids, etc.

### BRIEF DESCRIPTION OF THE DRAWINGS

Other features, details, and advantages will become apparent upon reading the detailed description below, and upon analyzing the appended drawings.

FIG. 1 respectively illustrates the HPLC-DAD profile (280 nm characteristic of phenol compounds) of a dry crude extract of fermented grape marc from the Cabernet Franc grape variety (on the 12th day of fermentation) (top chromatogram) and of a dry crude extract of unfermented grape marc (fermentation D0) from the Cabernet Franc grape variety (bottom chromatogram).

FIG. 2 respectively illustrates the HPLC-DAD profile (280 nm characteristic of phenol compounds) of a dry ethanolic extract of fermented grape marc from the Cabernet Franc grape variety (on the 12th day of fermentation) (top chromatogram) and of a dry ethanolic extract of unfermented grape marc (fermentation D0) from the Cabernet Franc grape variety (bottom chromatogram).

FIG. 3 illustrates anti-inflammatory activity, by measuring the percentage inhibition of the 15-lipoxygenase enzyme (15-LOX) by a dry ethanolic extract of fermented grape marc from the Cabernet Franc grape variety (on the 12th day of fermentation) (right histogram) and by a dry ethanolic extract of unfermented grape marc (fermentation D0) of the Cabernet Franc grape variety (left histogram) dissolved at 50 μg/mL.

FIG. 4 illustrates antioxidant activity, by measuring the percentage inhibition of the oxidation of the 2,2-diphenyl-1-picrylhydrazyl radical (DPPH) by a dry ethanolic extract of fermented grape marc from the Négrette grape variety (on the 12th day of fermentation) (right histogram) and by a dry ethanolic extract of unfermented grape marc (fermentation D0) from the Négrette grape variety (left histogram) dissolved at 50 μg/mL.

FIG. 5 illustrates the antidiabetic activity, by measuring the percentage inhibition (IC50 μg/mL) for the enzymes alpha-amylase (see the two leftmost histograms) and alpha glucosidase (see the two rightmost histograms), by a dry ethanolic extract of fermented grape marc from a mixture of Cabernet franc, Cabernet Sauvignon and Malbec (on the 5th day of fermentation) (see the dark gray histograms: fermentation D5) and a dry ethanolic extract of unfermented grape marc from a mixture of Cabernet franc, Cabernet Sauvignon and Malbec (see the light gray histograms: fermentation D0). The extracts were dissolved at 50 μg/mL.

### DETAILED DESCRIPTION

The drawings and description below may not only serve to provide a better understanding of the invention, but where appropriate may also contribute to its definition. The following examples make reference to FIGS. 1 to 5.

### EXAMPLES

### Example 1

### Preparation of Fermented Grape Marc

Four grape marcs which respectively come from the Cabernet Franc, Malbec, Négrette and Syrah black grape varieties are respectively fermented according to the method of the invention. The preparation method described in this example is carried out in particular by adding kombucha to the grape marc infusion. The first step described below concerns the preparation of the kombucha.

### Preparation of a Kombucha With 15 Days of Fermentation

A 1 L quantity of kombucha is prepared from an infusion of Ceylon black tea according to the following protocol.

In order to allow for evaporation during infusion and cooling, slightly more than 1 L of water is heated to approximately 35° C. then 70 g of sucrose is added, to obtain sugar water at a concentration of approximately 70 g/L. The temperature is increased to 80° C. (+/−2° C.) then 10 g of black tea is added to obtain a concentration of approximately 10 g/L of tea. The tea, contained in a gauze bag, is allowed to infuse for approximately 15 minutes while stirring regularly, then the gauze bag containing the tea is removed to obtain the tea infusion. The tea infusion is then left to cool down to a temperature below 30° C. (preferably around 25° C.).

In a glass beaker type of container, the following ingredients are added:
970 mL of sweetened black tea infusion as obtained in the step described above,
10 mL of cider vinegar,
20 g of scoby,
20 mL of kombucha.

The first time the kombucha was prepared, the Inventors purchased a black tea scoby on the site www.je-mange-vivant.com. The kombucha used (in preparing the kombucha) is a black tea kombucha marketed by the Germline company.

The container is covered with gauze.

Fermentation is carried out for a period of 15 days, protected from light and at a temperature of around 25° C.

At the end of fermentation, approximately 850 mL of kombucha is obtained. Part of this kombucha will be used in the method for preparing fermented grape marc while another part of this kombucha will be retained for use in preparing a new kombucha. This kombucha will be referred to below as “15-day fermentation kombucha”.

Similarly, at the end of fermentation, approximately 100 g of scoby is obtained. The kombucha baby (new scoby) is separated from the kombucha mother (original scoby). Part of the new scoby will be used in the method for preparing fermented grape marc while another part will be retained for use in preparing a new kombucha. This scoby will be referred to in the following as “scoby obtained from a 15-day fermentation kombucha”.

The scoby obtained from a 15-day fermentation kombucha mainly comprises the bacteria Komagataeibacter rhaeticus, Gluconacetobacter, and Gluconobacter, and the yeasts Brettanomyces and Schizosaccharomyces. This scoby is close to the one identified in the document by Villarreal-Soto et al.¹

### Preparation of the Grape Marc Infusion

The method of the invention described below is carried out with grape marc from the Cabernet Franc grape variety. However, the same method is applied using grape marc from the Malbec, Négrette and Syrah grape varieties respectively. Cabernet Franc, Malbec, Négrette and Syrah grape marc are in the form of a very friable solid residue.

In this example, dry residue from grape marc is used in particular.

1.2 L of water is heated to approximately 35° C., then 50 g of sucrose is added in order to obtain sugar water with a concentration of approximately 40 g/L. The temperature is increased to approximately 80° C. (+/−2° C.), then 150 g of dry grape marc is added in a gauze bag so as to obtain a ratio of dry matter/water that is approximately 70 g of dry grape marc per liter of water. The grape marc in contact with the sugar water is left to infuse for approximately 15 minutes at a temperature of approximately 80° C. with continuous automated stirring at 1200 rpm in a 2 liter beaker. The grape marc infusion is separated from the gauze bag containing the insoluble grape marc residue.

The grape marc infusion thus obtained is left to cool down to a temperature below 30° C. (preferably around 25° C.). Approximately 0.5 g/L of ammonium sulfate ((NH₄)₂SO₄) is added to the cooled grape marc infusion.

### Fermentation of the Grape Marc Infusion

In a glass beaker type of container, the following ingredients are added:
980 mL of grape marc infusion obtained in the step described above,
20 mL of 15-day fermentation kombucha (obtained in the step described above),
20 g of scoby obtained from a 15-day fermentation kombucha (obtained in the step described above).

The container is covered with gauze.

Fermentation of the grape marc infusion is carried out for a period of 12 days, protected from light and at a temperature of approximately 25° C., without stirring.

At the end of fermentation, approximately 850 mL of fermented grape marc infusion is obtained. The scoby is removed from the infusion in order to collect the fermented grape marc infusion without scoby. The fermented grape marc infusion without scoby is grape marc kombucha, and more specifically Cabernet Franc grape marc kombucha.

Malbec, Négrette and Syrah grape marc kombucha are obtained in the same manner as the Cabernet Franc grape marc kombucha.

The amounts defined above are adjusted in order to prepare 20 to 50 L of grape marc kombucha.

Various tank geometries may be used, with height/diameter ratios varying from 0.7 to 1.4. The stirring devices may operate intermittently or continuously.

Fermentations may be carried out with or without the addition of air by microbubbling in the liquid using a device such as a ceramic or aquarium bubbler.

### Preparation of Dry Crude Extracts of Fermented Grape Marc

Each of the fermented grape marc infusions obtained in the previous step (amount obtained=850 mL) is centrifuged at 10,000 rpm for 10 minutes.

The supernatant present on the surface of each of the infusions at the end of centrifugation is collected. A sample of each of these supernatants, approximately 300 mL, is placed in a 500 mL flask for evaporation at 35° C./30 rpm in a vacuum of 10 mbar±2 mbar.

The dry crude extracts obtained are brown and viscous in appearance and each has a mass of approximately 2 to 3.5 g, from 300 mL of supernatant. The yield varies depending on the grape variety and the fermentation conditions (method parameters, grape variety, duration, etc.).

Part of the dry crude extracts thus obtained is stored at a temperature of −20° C. until used for analysis of their biological activities.

Another part of the dry crude extracts is transformed into dry ethanolic extracts.

As an indication, approximately 10 to 20 g of dry crude extract is obtained per liter of evaporated grape marc kombucha. This yield varies depending on the grape variety and the fermentation conditions (method parameters, grape variety, duration, etc.).

### Preparation of Dry Ethanolic Extracts of Fermented Grape Marc

The dry crude extracts used to prepare the dry ethanolic extracts may be fresh dry crude extracts (not frozen) or frozen extracts. If the dry crude extract has been frozen, it must be defrosted first.

20 mL of absolute ethanol is added to 1 g of dry crude extract (fresh or thawed). The obtained mixture is stirred for approximately 1 hour using a magnetic stir plate. The ethanol mixture obtained at the end of stirring is collected and then filtered twice with 0.22 μm filter paper. The ethanol solution obtained after the two filtrations is evaporated at 35° C. at a rotation speed of 200-250 rpm/min until a dry (ethanolic) extract is obtained.

The dry ethanolic extracts are brown/red in appearance and not very viscous. They each have a mass of approximately 100 to 300 mg.

For dry ethanolic extracts, the yields vary from 10 to 30% compared to the dry crude extract.

### Example 2

### Chemical Profile and Study of the Biological Activities of Dry Extracts of Fermented Grape Marc

The chemical profile and biological activities of the dry extracts (crude and ethanolic) were studied in vitro via:
the study of their phenolic profiles by HPLC-DAD,
their anti-inflammatory activity on 15-LOX,
their antioxidant activity by DPPH,
their antidiabetic activity (alpha-glucosidase and alpha-amylase).

The studies were carried out on dry extracts (crude and ethanolic) of kombucha from Cabernet Franc, Malbec, Négrette and Syrah grape marc or blends thereof. The results obtained are similar for the four types of grape varieties as well as for their blends.

Furthermore, the dry extracts (crude and ethanolic) of Cabernet Franc grape marc kombucha were compared with dry extracts (crude and ethanolic) of unfermented grape marc.

### Preparation of Dry Extracts (Crude and Ethanolic) of Unfermented Grape Marc

The grape marc infusion is prepared according to the method described above (see paragraph “Preparation of grape marc infusion”), with an addition of ammonium sulfate after cooling.

The dry extracts of unfermented grape marc are prepared from the grape marc infusion (unfermented) according to a protocol identical to the one described above for the dry crude and ethanolic extracts of fermented grape marc from fermented grape marc infusion. Thus the dry crude extracts of unfermented grape marc are firstly prepared from the unfermented grape marc infusion, then the dry ethanolic extracts are prepared from the dry crude extracts.

The dry crude extracts of unfermented grape marc are brown/red in appearance and viscous. They each have a mass of approximately 10 to 20 g, obtained from a liter of marc infusion.

The dry ethanolic extracts of unfermented grape marc are brown/red in appearance and viscous. They each have a mass of approximately 100 to 300 mg, obtained from 1 g of dry crude extract of grape marc.

### Phenolic Profiles by HPLC-DAD

The phenolic profiles of the dry crude and ethanolic extracts of grape marc kombucha were analyzed by reverse-phase HPLC-DAD. Detection of the compounds was carried out at a wavelength of 280 nm.

In order to analyze them, the dry extracts were dissolved in a water/acetonitrile mixture at 20 mg/ml.

The mobile phase is composed of A: Acidified water (acetic acid, pH 2.65) and B: mixture of acidified water-acetonitrile (20:80 v/v) with an elution rate of 1.2 ml/min.

The samples were eluted by a gradient from 12% B to 30% B for 35 min, from 30% B to 50% for 5 min, from 50% B to 88% B for 5 min, and finally from 88% B to 12% B for 15 min. The column used is C18 (Phenomenex), 25 cm×4.6 mm with a particle size of 5 μm.

The dry crude and ethanolic extracts more particularly studied in this example are those obtained from the Cabernet Franc grape variety.

The dry crude extract of grape marc with 12 days of fermentation is compared with the dry crude extract of unfermented grape marc (FIG. 1).

The effects of fermentation on the HPLC-DAD profile without ethanol extraction are shown in FIG. 1. “Fermentation D0” indicates that the extract is an unfermented grape marc extract. The chromatogram close to the baseline is the one for the dry crude extract of unfermented grape marc (fermentation D0) while the one at the top is the one for the dry crude extract of fermented grape marc at D12. The formation of new compounds during the fermentation stage is clearly apparent in FIG. 1.

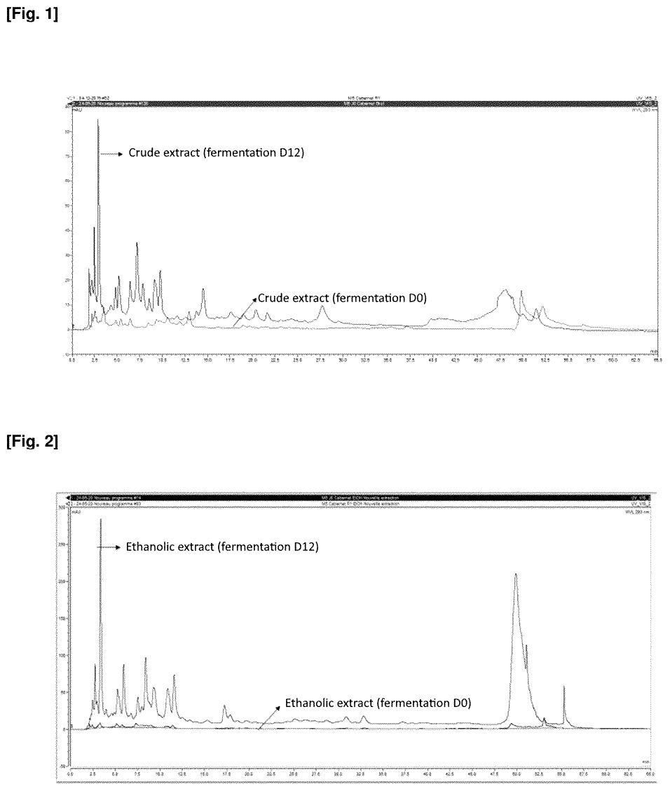

In the same manner, the dry ethanolic extract of grape marc with 12 days of fermentation is compared with the dry ethanolic extract of unfermented grape marc (see FIG. 2).

The effects of fermentation on the HPLC-DAD profile with ethanol extraction are illustrated in FIG. 2. The chromatogram close to the baseline is the one for the dry ethanolic extract of unfermented grape marc (fermentation D0) while the one at the top is the one for the dry ethanolic extract of fermented grape marc at D12. FIG. 2 clearly shows the formation of new compounds during the fermentation stage.

### Anti-Inflammatory Activity on 15-LOX

The dry extracts (crude and ethanolic) of grape marc kombucha were solubilized in pure DMSO then diluted to reach a concentration of 50 μg/mL and 1% DMSO, and were tested for their inhibitory activity on the 15-lipoxygenase enzyme.

The dry ethanolic extract of fermented grape marc more particularly studied in this example (on the 12th day of fermentation) comes from the Cabernet Franc grape variety (1 g/L) and is compared with the dry ethanolic extract of the same grape marc, unfermented (fermentation D0).

The results obtained are illustrated in FIG. 3. This figure clearly shows the improved anti-inflammatory activity of the ethanolic extract of fermented grape marc versus the ethanolic extract of unfermented grape marc.

### Antioxidant Activity by DPPH

The antioxidant activity of grape marc kombucha was evaluated by determining the anti-radical activity using the “2,2-diphenyl-1-picrylhydrazyl” or DPPH radical method.

It is expressed by the percentage inhibition of the oxidation of DPPH radicals present in the reaction medium, by the ethanolic extract dissolved in DMSO at 50 μg/mL.

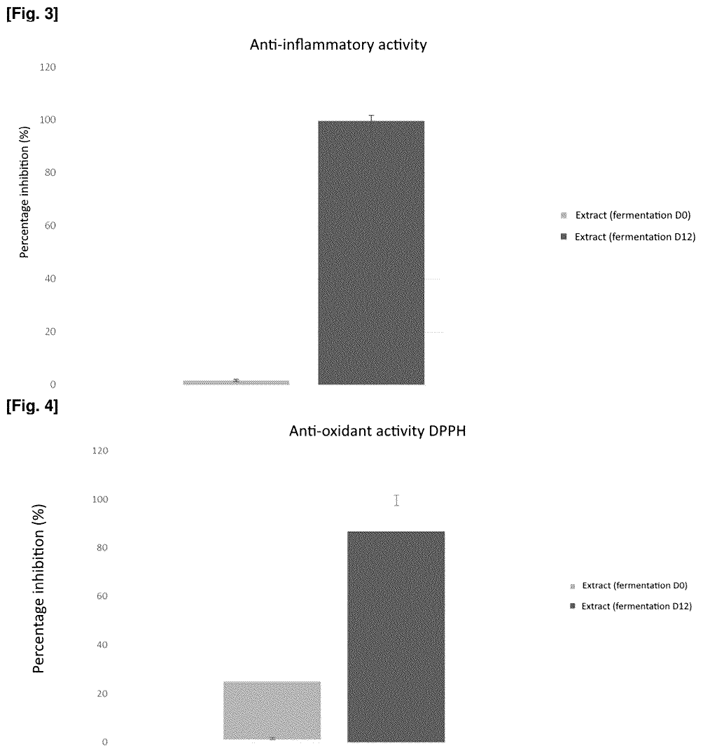

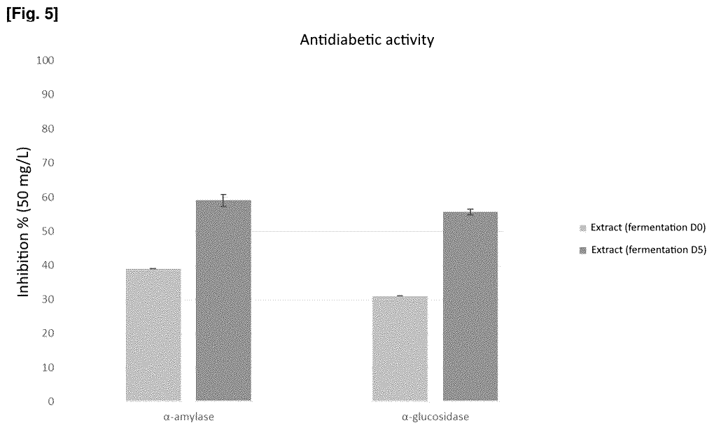

The results obtained are illustrated in FIG. 4 for the Négrette grape variety. The improvement in the antioxidant activity of the ethanolic extract of grape marc after fermentation (on the 12th day of fermentation) compared to the ethanolic extract of unfermented grape marc (fermentation D0) is clearly apparent in this figure.

### Antidiabetic Activity

The dry ethanolic extracts of grape marc kombucha from a mixture of Cabernet franc, Cabernet Sauvignon and Malbec on the 5th day of fermentation (fermentation D5) were diluted in pure DMSO, to reach a concentration of 50 μg/mL. These extracts were compared to ethanolic extracts of unfermented grape marc (fermentation D0) from a mixture of Cabernet franc, Cabernet Sauvignon and Malbec and were tested for their inhibitory activity respectively on the alpha-glucosidase enzyme (see FIG. 5, the two histograms on the right) and the alpha-amylase enzyme (see FIG. 5, the two histograms on the left). The dark gray histograms represent the dry ethanolic extract of grape marc kombucha on the 5th day of fermentation while the light gray histograms represent the dry ethanolic extract of unfermented grape marc (fermentation D0). The improvement in the antidiabetic activity of the ethanolic extract of grape marc on the 5th day of fermentation compared to the ethanolic extract of unfermented grape marc is clearly apparent from FIG. 5, and this is true for both enzymes (alpha-glucosidase and alpha-amylase).

### LIST OF CITED DOCUMENTS

### Non-Patent Literature

For reference, the following non-patent element is cited:
nplcit1: Villarreal-Soto S A, Bouajila J*, Pace M, Leech J, Cotter P D, Souchard J P, Taillandier P. Beaufort S. Metabolome-microbiome signatures in the fermented beverage, Kombucha.

1-11. (canceled)

12. A method for preparing fermented grape marc, comprising the following steps:
preparing a grape marc infusion by placing grape marc in contact with water and possibly with a source of sugar chosen among sucrose, glucose syrup, or concentrated grape must;
placing the grape marc infusion in contact with a scoby (also called “kombucha microbial consortium”) and possibly with kombucha; and
fermenting the grape marc infusion for a period ranging from 5 to 35 days, and preferably from 7 to 12 days.

13. The method according to claim 12, wherein, at the end of fermentation, the scoby is removed from the infusion in order to recover the fermented grape marc infusion without the scoby, said fermented grape marc infusion from which the scoby has been removed also being called “grape marc kombucha”.

14. The method according to claim 12, wherein the fermented grape marc infusion from which the scoby has been removed is centrifuged or filtered.

15. The method according to claim 13, wherein the fermented grape marc infusion from which the scoby has been removed, optionally centrifuged or filtered, is evaporated or freeze-dried in order to obtain a dry crude extract of fermented grape marc, said dry crude extract of fermented grape also able to be called “dry crude extract of grape marc kombucha”.

16. The method according to claim 15, further comprising the following steps:
solubilizing the dry crude extract of grape marc kombucha in ethanol,
stirring the ethanol solution,
filtering the ethanol solution,
evaporating the filtered ethanol solution until a dry ethanolic extract is obtained, also called “dry ethanolic extract of grape marc kombucha”.

17. The method according to claim 12, wherein:
the scoby comes from a kombucha having 14 to 20 days of fermentation, preferably 15 to 17 days of fermentation,
the kombucha is a kombucha having 14 to 20 days of fermentation, preferably 15 to 17 days of fermentation,
the grape marc infusion has a concentration of “grape marc” dry matter per liter of water ranging from 20 to 150 g/L, preferably from 50 to 80 g/L, said water optionally comprising a source of sugar chosen among sucrose and/or concentrated grape must.

18. The method according to claim 12, wherein the grape marc, placed in contact with water in order to prepare the grape marc infusion, is in the form of a fresh solid residue, frozen solid residue, or dry solid residue.

19. The method according to claim 12, wherein the grape marc infusion is prepared according to the method comprising the following steps:
optionally, in a preliminary step, adding a source of sugar chosen among sucrose, glucose syrup, or concentrated grape must, to water previously heated to a temperature ranging from 30° C. to 40° C., in order to achieve a concentration ranging from 30 to 50 g/L of sugar per liter of water,
adding grape marc to water previously heated to a temperature ranging from 70° C. to 90° C., said water possibly being water prepared in the previous step (namely water comprising a source of sugar), so as to obtain an infusion having a concentration ranging from 20 to 150 g/L, preferably from 50 to 80 g/L, of “grape marc” dry matter per liter of water, said water optionally comprising a source of sugar,
maintaining the temperature of the infusion at a temperature ranging from 70° C. to 90° C., for a period ranging from 10 to 20 minutes,
filtering the grape marc infusion in order to separate it from the marc remaining in solid form,
cooling the filtered grape marc infusion to a temperature ranging from 20 to 35° C.,
optionally, adding to the filtered grape marc infusion a nitrogen source chosen among an organic nitrogen salt or ammonium salt, at a quantity ranging from 0.2 to 0.8 g/L of nitrogen source per liter of grape marc infusion.

20. A fermented grape marc obtainable according to the method of claim 12.

21. A dietary supplement, comprising a grape marc obtainable or obtained according to the method of claim 12, wherein said dietary supplement is able to be in the form of a solid or a liquid.

22. A nutricosmetic or in dermocosmetic product comprising comprising a grape marc obtainable or obtained according to the method of claim 12.
